(19) United States
(12) Patent Application Publication
(10) Pub. No.: US 2006/0228277 A1
(43) Pub. Date: Oct. 12, 2006

(54) Revolutionary toothbrush cleaner

(76) Inventor: Opal Marie Duncan, Winnipeg (CA)

Correspondence Address: Opal Duncan, 491 Lindenwood Dr. W, Winnipeg, MB, R3P-2P8, CA

(21) Appl. No.: 11/294,804

(22) Filed: Dec. 7, 2005

(30) Foreign Application Priority Data
Mar. 23, 2005 (CA) 2,503,605

(51) Int. Cl. A61L 2/18

(52) U.S. Cl. 422/300

(57) ABSTRACT

My invention is a product that can clean your toothbrush. The inventions intention is to promote healthy teeth and gums and to kill bacteria before they get in your mouth. By using the solution in the toothbrush cleaner, when the bristles portion is submerged into the liquid it can kill 99.9% of bacteria. It has a surrounding fin system that cleans your toothbrush bristles and kills bacteria and removes food and build-up from your toothbrush bristles. It also has four off set fins to dry excess liquid. It has two filter opening the upper is to pour solution into. The bottom filter is to drain liquid from the container. The base is a free standing container and can be removed for your convenience. It can also be used for traveling purposes. There is also a toothbrush holder that can hold four toothbrushes.

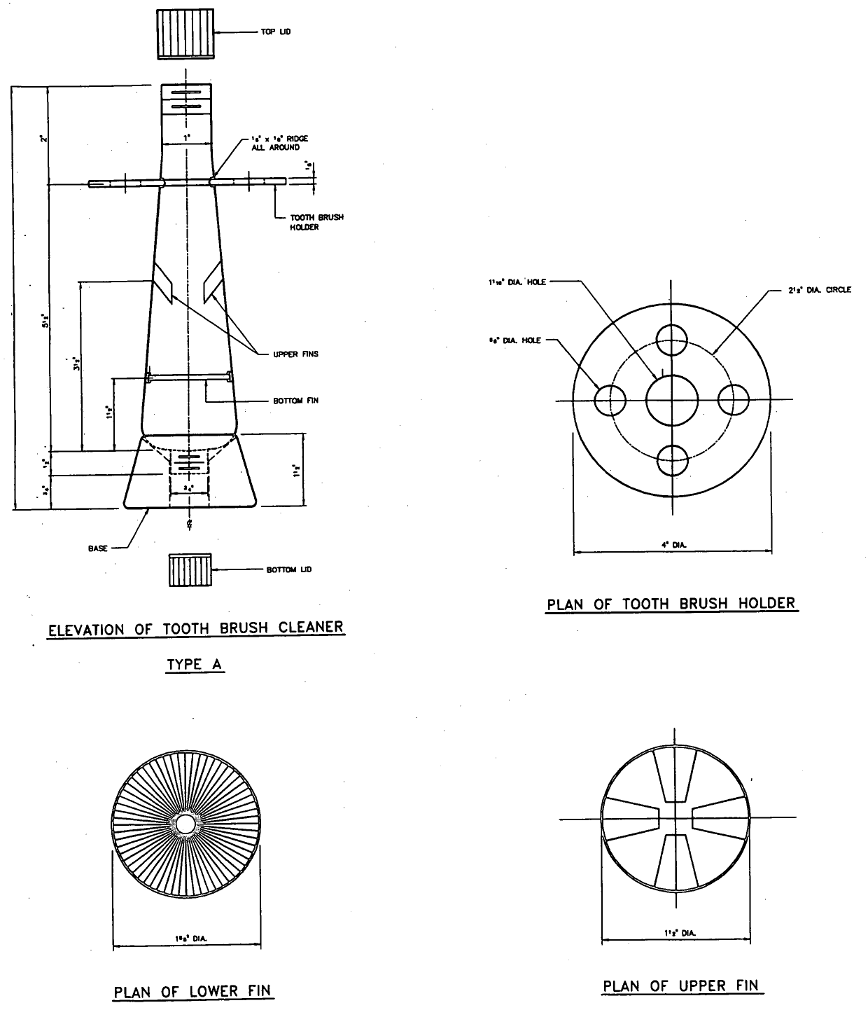

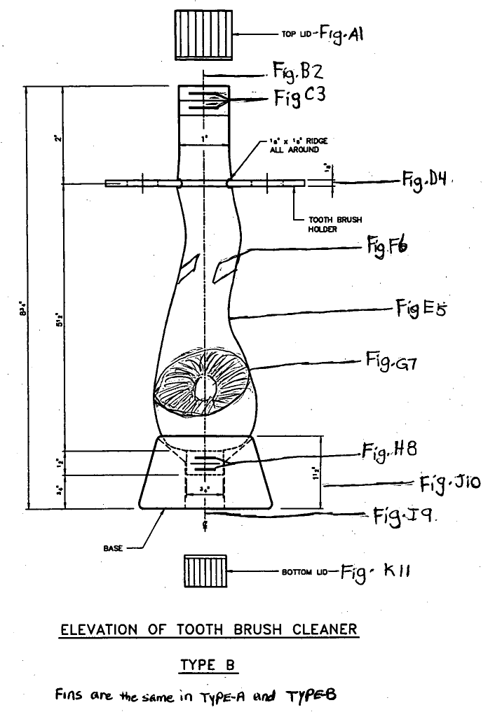

### SUMMARY OF INVENTION

Figure A and C are locked together to prevent spills. Figure B is to poor solution into the container. Figure D is to store toothbrushes when not cleaning. Figure E is the container Figure F is to dry or take excess solution from the toothbrush cleaner. Figure G Is used to wash your toothbrush Bristol free of any food or bacteria. Figure H and K are to prevent solution from spilling. FIG. I is funnel shaped to drain solution from container. Figure J's job is to keep container sturdy and firm.

Figure A: top lid Figure G: cleaning fins

Figure B: top opening Figure H: bottom ridge

Figure C: top ridge Figure I: bottom opening

Figure D: toothbrush holder Figure J: base

Figure E: cleaning container Figure K: bottom lid

Figure F: upper left fins

### BACKGROUND OF INVENTION

The present invention relates to a toothbrush cleaner for cleaning toothbrushes. The inventions intension is to promote healthy teeth and gums to kill bacteria before they get in your mouth. When the bristles portion is submerged into the liquid it can kill 99.9% of bacteria. It has a system that can dry excess liquid It also kills bacteria, removes food particles and build-up from your toothbrush bristles. It also has a draining system to let liquid out and a no spill system. The base can be removed into a free standing container or used for traveling purposes. “When was the last time you cleaned your toothbrush”. Imagine having a clean toothbrush anytime of the day.

### BRIEF DESCRIPTION

A) Top lid: the top lid is a child locked safe, safe to travel with and no spills.

B) Top opening: this opening of the container allows you to pour liquid solution in and to submerge toothbrush bristles into your solution.

C) Top ridge: the top lid and the ridge work together to ensure that the container is sealed from spills.

D) Toothbrush holder: the toothbrush holder is to hold your toothbrush in before you clean your toothbrush bristles. It is portable and removable when traveling and when it is not needed.

E) The cleaning container: the container contains the fins; you can put your toothbrush and solution in the container in order to have a clean toothbrush. It also has a ridge on the outside for your toothbrush holder.

F) Upper fins: the purpose of the upper fins is to dry excess liquid or solution from your toothbrush bristles.

G) Cleaning fins: the cleaning fins have four separate over lapping, off-set fins or surrounding fins with a space in the middle to receive the toothbrush bristles, the fins purpose is to clean food and build up from your toothbrush bristles when a sterilizer solution or mouthwash like solution is pour in the container it also kills bacteria from your toothbrush bristles, just place your toothbrush bristles in the solution and move in an upward and a downward motion against the fins to remove food, build-up and bacteria.

H) Bottom ridge: the bottom ridge works with the bottom cap to insure container from spilling.

I) the Bottom opening: the bottom opening is to drain the liquid solution from the container.

J) The toothbrush cleaner base: the base of the container is a free standing base to keep the container sturdy and strong.

K) Bottom cap: the bottom cap and bottom ridge works together So you can have a no spill container.

L) Toothbrush holder: the toothbrush holder can hold up to four toothbrushes. It is also portable and removable. Clean your toothbrush whenever you need to or just hold it in the toothbrush holder.

What I claim as my invention is:

1. that this revolutionary toothbrush cleaner which has four over-lapped, off-set fins or surrounding fins to clean your toothbrush bristles, and four upper fins to dry excess liquid from toothbrush bristles. The circular fins are inside the container with a space in the middle. It has pointy sharp edges to clean your toothbrush and fin liked shaped to dry excess water. The purpose of the fins is to remove food, build-up and bacteria from your toothbrush. When the bristle portion of the toothbrush is submerged into the liquid you can move your toothbrush in an upward and downward motion. The fins work between the bristles of your toothbrush to remove food build up and bacteria with the aid of a sterilizing solution or mouthwash like solution to kill bacteria on your toothbrush bristles. The container is a free standing child lock safe, it is ready to use any time! It has two filter openings: the upper filter is to pour solution or liquid into the container and to submerge your toothbrush bristles into. The bottom filter is to drain liquid solution from the container. It also has a toothbrush holder which is removable and portable.
